(19) United States
(12) Patent Application Publication
(10) Pub. No.: US 2007/0142745 A1
(43) Pub. Date: Jun. 21, 2007

(54) Method of procuring birth tissue

(76) Inventor: Timothy R. Brahm, Germantown, TN (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 11/314,356

(22) Filed: Dec. 21, 2005

(51) Int. Cl. A61B 10/00

(52) U.S. Cl. 600/562

(57) ABSTRACT

A method of procuring birth tissue which includes the following steps; identifying a suitable birth tissue donor, scheduling a birth procedure, providing a trained technician to procure birth tissue at the birth procedure and procuring birth tissue at the birth procedure. The method of procuring birth tissue is thus based upon the provision of a trained technician. This trained technician will typically be trained by a third party other than the hospital, clinic or medical staff primarily responsible for the health care provided the birth tissue donor at the birth procedure. The trained technician may be trained in some or all of the following skills; identifying a suitable birth tissue donor, obtaining consent for a birth tissue donation and following applicable tissue procurement regulations before, during and after the birth tissue procurement. In addition, the trained technician may be trained in the following skills; directing the persons primarily responsible for the health care provided to the birth tissue donor to maximize the quality of the donated birth tissue, providing initial treatment to the birth tissue procured, beginning the manufacturing process of products from the birth tissue, testing the birth tissue, measuring the birth tissue, packaging the birth tissue and shipping the birth tissue to a facility for further processing.

### TECHNICAL FIELD

The present invention is directed toward a method of procuring birth tissue and more particularly toward the provision of a skilled and trained technician who is not typically a member of a hospital staff to procure birth tissue at a planned birth event.

### BACKGROUND OF THE INVENTION

Over four million births occur in the United States each year. Several organic substances are byproducts of childbirth. These substances are referred to collectively as “birth tissue” herein. A synonym for birth tissue is “afterbirth.” Properly collected birth tissues holds substantial promise for the development of medical products and therapeutic substances useful in the implantation, tissue culture and related industries.

The useful tissues, fluids and cells which are included in birth tissue are derived from many sources. For example, the umbilical cord contains:

1. Cord blood rich with pluripotent, hematopoetic stem cells that can be used in place of bone marrow transplantation and for other tissue regenerative applications such as gene transfer, trauma tissue regeneration, stroke regeneration, cardiac regeneration, vascular regeneration, bone formation and wound healing.

2. The Wharton's Jelly which is rich in mesenchymal stem cells, hyaluronic acid and various growth factors which are useful for tissue and organ regeneration, repair and preservation.

3. Collagen useful for tissue augmentation, reconstruction, regeneration and also as a delivery mechanism for other biologics.

4. Two arteries that may be engineered for cardiac bypass procedures and other forms of peripheral and venous reconstruction and as biologic tissue conduits such as balloons and stents.

5. One vein useful for bypass surgery, biologic tissue patches, nerve conduits, as an access structure for dialysis and vascular or spinal implants.

Similarly, the placenta contains:

1. Additional cord blood not found in the cord during birth.

2. Mesenchymal stem cells, growth factors and other cells and enzymes useful for tissue engineering and regeneration.

3. Large quantities of collagen.

Birth tissue, if collected at all, is typically collected by untrained hospital staff and or the patient's OB/Gyn. Collection efforts are usually limited to one of the birth tissues of interest and not toward a group of tissues to be processed into individual products. Most birth tissue collections are limited to cord blood for storage and use at a later date. The cord blood companies provide a “kit” for the staff or physician to utilize for recovery and shipping during delivery of the baby The blood or other tissue may be collected, labeled and sent to a processing or storage site. The processing/storage facilities typically quarantine the recovered blood or tissues prior to production or processing and ultimately store or distribute the prepared products.

Alternatively, hospital/delivery room staff may directly recover birth tissue after performing a birth procedure. Certain problems exist with the use of hospital staff for the proper recovery of birth tissues. In particular, it is usually critical to obtain and preserve the human cells and tissue from the birth tissue donor during the somewhat chaotic and emotional birth process. Often during birth, the time and energy focus of the hospital staff are appropriately directed toward the newborn child and cannot be expected to be focused on collecting and processing the afterbirth. Due to the time and environment sensitive nature of the tissue and cells as transplant material, any increase in the duration of tissue collection activities from delivery to preservation will result in a reduction of cell viability and quality. If untrained personnel are responsible for the critical functions unique to this recovery, the cells and tissues will be severely diminished at best, probably unusable and quite easily unsafe for transplantation. In addition, hospital staff are often not well trained in the techniques necessary to maximize the quality of the birth tissue procured or in the preliminary treatment and packaging steps necessary to maximize the yield of useable birth tissue. For example, only trained and certified recovery specialists will know how and when to appropriately culture the potentially transplantable blood and tissue during recovery. Thus, reliance on hospital staff may result in the collection of less than optimum quality birth tissue or the collection of less than the maximum quantity of birth tissue.

The procurement of human tissue is a highly regulated activity. For example, 21 CFR Part 1271 includes federal regulations relating to human cells, tissues and cellular and tissue based products. These regulations, if not followed during donor qualification, tissue recovery, and processing, can render collected tissue ineligible for human clinical use. These regulations are very comprehensive in nature and require control and complete documentation of nearly every aspect of the process from recovery to implantation in order to ensure the quality of the final medical product and reduce the risk of transmission of communicable diseases. In most cases today, individuals involved in the process of procuring birth tissues have little or no understanding of these regulatory requirements placing any recovered tissue at risk of being unusable or subject to recall should resulting products reach distribution channels.

Thus, another problem with present birth tissue collection practices which rely upon hospital staff or on-call recovery agents arises from the complexity of the applicable regulatory framework and the near impossibility of complying with the regulatory framework without a great deal of training. Hospital staff have many duties in general and during a birth procedure in particular. Hospital staff are necessarily well trained in providing health care. It is, however, difficult and inefficient to also train hospital staff in compliance with present and future birth tissue collection regulations.

The present invention is directed toward overcoming one or more of the problems discussed above.

### SUMMARY OF THE INVENTION

One aspect of the present invention is a method of procuring birth tissue which includes the following steps; identifying and qualifying a suitable birth tissue donor, scheduling a birth procedure, providing a trained technician to procure birth tissue at the birth procedure and procuring birth tissue at the birth procedure.

The method of procuring birth tissue is thus based upon the provision of a trained technician. This trained technician will typically be trained by a third party other than the hospital, clinic or medical staff primarily responsible for the health care provided to the birth tissue donor at the birth procedure. The trained technician may be trained in some or all of the following skills; identifying a suitable birth tissue donor, obtaining consent for a birth tissue donation and following applicable tissue procurement regulations before, during and after the birth tissue procurement. In addition, the trained technician may be trained in the following skills; directing the persons primarily responsible for the health care provided to the birth tissue donor to maximize the quality of the donated birth tissue, providing initial treatment to the birth tissue procured, beginning the manufacturing process of products from the birth tissue, testing the birth tissue, measuring the birth tissue, packaging the birth tissue and shipping the birth tissue to a facility for further processing.

Prior to the scheduling of a birth event, the trained technician may provide sufficient education to the birth tissue donor to allow the donor to provide informed consent, to interview the birth tissue donor to satisfy regulatory requirements and to ensure donor tissue quality and safety, and obtain a blood sample used for testing for relevant communicable diseases. The trained technician may be primarily responsible for obtaining the consent, blood sample and medical and social history of the donor after suitable education efforts have been rendered.

Normal childbirth with a vaginal delivery of a baby is a relatively non-sterile procedure. Therefore, birth tissue collected during normal childbirth is often contaminated. Thus, the ideal birth procedure for the procurement of sterile birth tissue is a planned (non-emergency) cesarean section procedure or emergency procedure performed in an operating room under sterile conditions. Thus, the step of identifying a suitable birth tissue donor by the trained technician may include collecting serological testing samples from the patient, obtaining a medical/social history from the patient and identifying patients who, for specific medical reasons, must schedule a cesarean section procedure for delivery of a baby.

During the birth procedure, typically a planned cesarean section, the trained technician may direct the actions of the birth procedure staff to maximize the quality or quantity of the birth tissue procured. Upon procurement of birth tissue, the trained technician may take some or all of the following steps to assure the usefulness of the birth tissue collected; treating the birth tissue with a chemical or pharmaceutical product, applying a tissue culture medium and marking the birth tissue with a tissue marker. In addition, the trained technician may test and measure certain physical and biological attributes of the birth tissue and take steps to minimize the duration the collected tissue is maintained in a condition of warm ischemia. The trained technician may also be primarily responsible for dissecting the different tissues, preserving each individual tissue specific to its characteristic or future function and packaging the birth tissue for shipment to a manufacturing laboratory or several different manufacturing laboratories, processing facilities and/or storage facilities and shipping the birth tissue to a manufacturing laboratory or several different manufacturing laboratories, processing facilities and/or storage facilities.

At all times, the trained technician will be the person primarily responsible for assuring that all applicable regulatory requirements relating to birth tissue donation and recovery are met. Thus, the trained technician's regulatory compliance duties may include supervising the physical collection of the birth tissue as well as preparing and filing all necessary documentation in accordance with applicable regulations.

### BRIEF DESCRIPTION OF THE DRAWINGS

FIG. 1 is a flow chart showing certain steps undertaken by a trained technician prior to a birth procedure in accordance with one aspect of the present invention;

FIG. 2 is a flow chart showing certain steps taken by a trained technician during and after a birth procedure in accordance with one aspect of the present invention.

### DETAILED DESCRIPTION OF THE INVENTION

The present invention includes a comprehensive method of procuring birth tissue. In lieu of relying on hospital staff or contacting an on-call tissue recovery agent, procurement efficiency may be increased and overall costs may be reduced by relying upon a specifically trained technician to procure birth tissue at a birth procedure. The trained technician may be trained in both the mechanics of birth tissue recovery and applicable regulatory matters. Thus, the procurement of birth tissue using a specifically trained technician may enhance the level of quality of the tissue recovered, increased product yield and ensure regulatory compliance and tissue safety.

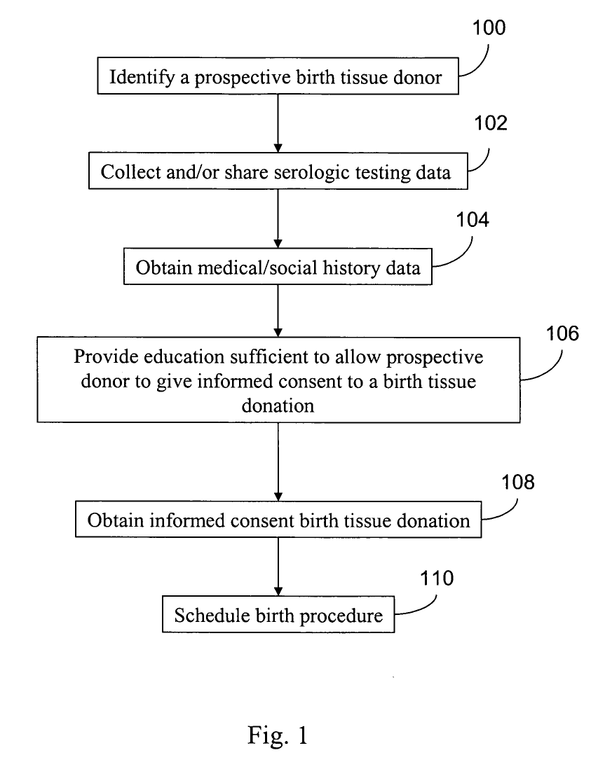

A normal, vaginal delivery of a child is not particularly sterile. Thus, the trained technician of the present invention will typically identify prospective donors as mothers who must, for other medical reasons, schedule a cesarean section birth procedure in advance. As is shown on FIG. 1 and described below, the trained technician may engage in several steps prior to the birth procedure to assure the quality of the birth tissue procured, and furthermore to assure that all applicable regulatory requirements are met.

The trained technician responsible for implementing the methods described herein will typically be trained by a third party other than the hospital, clinic or birthing center primarily responsible for the health care provided to the birth tissue donor during the birth procedure. It is possible, however, that the trained technician will have multiple responsibilities with respect to the birth procedure. The trained technician may, for example, be trained with the methods disclosed herein as well as fulfilling the duties of an anesthesiologist or an operating room nurse.

The trained technician who will implement the methods described herein may be trained in at least one and preferably more than one or all of the following skills: identifying a suitable birth tissue donor, obtaining consent for a birth tissue donation from a birth tissue donor and following applicable tissue procurement regulations incident to the birth tissue procurement. In addition, the trained technician may be skilled in directing the persons or staff primarily responsible for the health care provided to the birth tissue donor at the birth procedure, thus providing for the maximization of the quality or quantity of the donated birth tissue. The trained technician may be trained in suitable methods of treating the birth tissue procured, beginning the manufacturing process of products ultimately to be derived from the birth tissue procured and testing or measuring a physical attribute of the birth tissue. In addition, the trained technician may be trained in dissecting and packaging the birth tissue procured, performing initial time, transportation and atmosphere sensitive preservation steps and shipping the birth tissue procured to a laboratory or manufacturing facility for further processing.

FIG. 1 depicts a flow chart of steps to be taken by the trained technician prior to the commencement of a birth procedure which steps are consistent with the present methods. Initially the trained technician will in conjunction with the primary health care providers identify a prospective birth tissue donor (step 100). As discussed above, a prospective birth tissue donor will primarily be identified as a prospective mother who must, for other medical reasons, schedule a cesarean section or other sterile delivery. Next, the trained technician may collect and/or share serologic testing data and or serological specimens needed to determine transplant suitability (step 102). Also, the trained technician may be primarily responsible for obtaining and reviewing a medical or social history from the prospective tissue donor (step 104). The technician may also utilize the time afforded by the planned delivery system to screen the potential donor with appropriate medical and regulatory staff to predetermine the donated tissue eligibility for recovery, processing and transplantation. Once a prospective birth tissue donor has been identified, the trained technician may be primarily responsible for providing education to the prospective donor sufficient to allow the donor to form and give informed consent to a birth tissue donation (step 106). Such education may be provided by direct discussion with the prospective donor during a pre-delivery office or hospital visit, through written or video materials, or through other conventional means of providing education concerning anticipated medical procedures. Subsequent to the provision of education to the prospective birth tissue donor, the trained technician may obtain consent from the donor for the procurement and use of birth tissue (step 108). In conjunction with the primary health care staff and the birth tissue donor, the trained technician may directly schedule or participate in the scheduling of the future birth procedure (step 110).

Each of the steps described above with respect to FIG. 1 must be performed and fully documented in accordance with applicable tissue collection regulations. It is the primary responsibility of the trained technician to assure that all steps are taken in accordance with applicable regulations and Standard Operating Procedures and that all necessary documentation is completed and submitted in compliance with regulatory requirements.

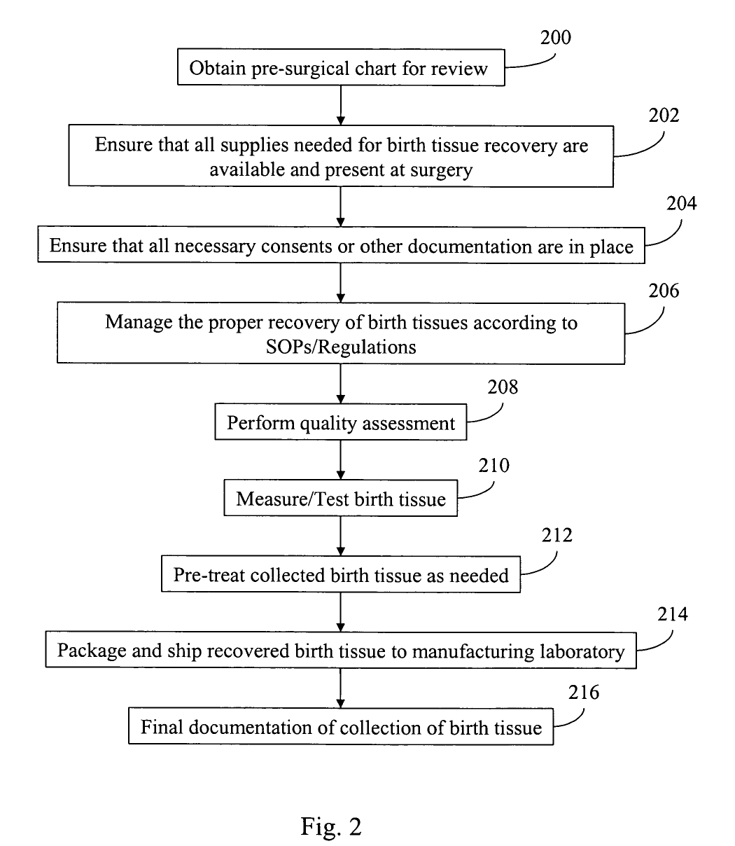

FIG. 2 depicts a flowchart of steps which may be taken by the trained technician immediately prior to, during and after the scheduled birth procedure. Initially, the trained technician may obtain pre surgical charts (step 200) and review the charts for medical concerns, for regulatory compliance purposes. Alternatively, the trained technician may forward the chart to a supervisor for such review. In addition, the trained technician may insure that all supplies necessary for birth tissue recovery are available and present in the operating room (step 202). This aspect of the trained technician's duties is different from and supplemental to the operating room staffs responsibilities which are typically limited to insuring that the supplies and materials necessary to complete the caesarian section are present in the operating room. Finally, prior to the commencement of surgery, the trained technician may insure that all necessary consents or other documentation required by Standard Operating Procedures (SOPs) or regulations are fully executed and properly filed (step 204).

During the actual birth procedure, typically a caesarian section, the trained technician may direct the primary health care providers as necessary to manage the proper recovery of birth tissues (step 206). The management of the proper recovery of birth tissues may include providing advice and direction concerning proper medical techniques and procedures useful to maximize the quality and quantity of the birth tissue recovered, preserve birth tissue sterility, or otherwise assure that quality tissue is collected. Management of the proper recovery of birth tissues may also include assuring that the requirements of any appropriate regulations or applicable SOPs are met.

Upon procurement of birth tissue, the trained technician may perform an initial quality assessment of the tissue (step 208). The initial quality assessment may include measurement of physical parameters of certain tissues, or the performance of initial qualitative tests upon the birth tissue (step 210). In addition, the trained technician may pre-treat collected birth cells and tissue as needed (step 212).

Immediate recovery of birth tissue by a trained technician as described above will allow for the maintenance and enhancement of birth tissue cellular viability to maximize the recovery of specific cells. Pretreatment of birth tissues with a chemical, compound or pharmaceutical to drive a specific reaction toward recovery of a specific end product is also possible. These end products could include but are not limited to specific trace elements, growth factors, enzymes or chemicals.

The presence of a trained technician at the birth procedure also allows the trained technician to take steps to stop other specific reactions in order to recover specific end products as described above. Thus, the pre-treatment step (step 212) may be used to preserve specific trace elements, growth factors, enzymes or chemicals which may be present at the time of birth but begin to break down immediately after birth.

The trained technician may also take steps to ensure that the duration the collected birth tissue is maintained in a state of warm ischemia is minimized to protect from cellular degradation.

The pre-treatment of collected birth tissue (step 212) may include, but is not limited to, the application of pharmaceuticals, chemicals, tissue culture media, antibiotics, tissue markers or other substances to the recovered tissues to provide for the enhancement of recovery or elimination of selected tissue products. After pre-treatment, the trained technician may be primarily responsible for the packaging and shipping of the recovered birth tissues to one or more manufacturing laboratories (step 214). The trained technician may have discretionary authority to divide the birth tissue as necessary and ship portions of the birth tissue to separate facilities allowing specific facilities to utilize their best efforts to provide and manufacture appropriate products from the recovered birth tissue.

The trained technician shall finally be responsible for post surgical documentation of the collection of birth tissue (step 216). The foregoing documentation may be prepared in compliance with all applicable regulations and SOPs.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

What is claimed is:

1. A method of procuring birth tissue comprising:
identifying a birth tissue donor;
scheduling a birth procedure;
providing a trained technician to procure birth tissue at the birth procedure; and
procuring birth tissue at the birth procedure.

2. The method of procuring birth tissue of claim 1 wherein the trained technician is trained by a third party other than the entity primarily responsible for the health care provided to the birth tissue donor at the birth procedure.

3. The method of procuring birth tissue of claim 2 wherein the trained technician is trained in at least one of the following skills:
identifying a suitable birth tissue donor;
obtaining consent for a birth tissue donation from a birth tissue donor;
following applicable tissue procurement regulations incident to birth tissue procurement;
directing the persons primarily responsible for the health care provided to the birth tissue donor at the birth procedure to maximize the quality of the donated birth tissue;
treating the birth tissue procured;
beginning the manufacturing process of products from the birth tissue procured;
testing the birth tissue procured;
measuring a physical attribute of the birth tissue procured;
packaging the birth tissue procured; and
shipping the birth tissue procured.

4. The method of procuring birth tissue of claim 2 wherein the trained technician is trained in each of the following skills:
identifying a suitable birth tissue donor;
obtaining consent for a birth tissue donation from a birth tissue donor;
following applicable tissue procurement regulations incident to birth tissue procurement;
directing the persons primarily responsible for the health care provided to the birth tissue donor at the birth procedure to maximize the quality of the donated birth tissue;
treating the birth tissue procured;
beginning the manufacturing process of products from the birth tissue procured;
testing the birth tissue procured;
measuring a physical attribute of the birth tissue procured;
packaging the birth tissue procured; and
shipping the birth tissue procured.

5. The method of procuring birth tissue of claim 3 further comprising:
providing sufficient education to the birth tissue donor prior to the commencement of the birth procedure, to allow the birth tissue donor to provide informed consent for the procurement of birth tissue; and
obtaining consent from the birth tissue donor to procure birth tissue, the consent being obtained prior to the commencement of the birth procedure.

6. The method of procuring birth tissue of claim 3 wherein the birth procedure is a planned caesarian section procedure.

7. The method of procuring birth tissue of claim 3 wherein the step of identifying a birth tissue donor comprises:
collecting serologic testing data; and
obtaining medical/social history data.

8. The method of procuring birth tissue of claim 3 further comprising performing a quality assessment of the birth tissue collected by the trained technician.

9. The method of procuring birth tissue of claim 3 further comprising treating the birth tissue collected by the trained technician.

10. The method of procuring birth tissue of claim 9 wherein the step of treating the birth tissue collected by the trained technician comprises at least one of the following steps:
treating the birth tissue with a chemical;
treating the birth tissue with a pharmaceutical product;
treating the birth tissue with a tissue culture medium; and
treating the birth tissue with a tissue marker.

11. The method of procuring birth tissue of claim 3 further comprising minimizing the duration the collected tissue is in a condition of warm ischemia.

12. The method of procuring birth tissue of claim 3 further comprising determining a size parameter of the birth tissue collected by the trained technician.

13. The method of procuring birth tissue of claim 3 further comprising:
packaging the birth tissue for shipment to a manufacturing laboratory; and
shipping the birth tissue to a manufacturing laboratory.

14. The method of procuring birth tissue of claim 3 further comprising documenting the collection of birth tissue in accordance with applicable regulations.

15. A method of procuring birth tissue comprising:
identifying a birth tissue donor;
scheduling a birth procedure;
providing a trained technician to procure birth tissue at the birth procedure;
directing the actions of the birth procedure staff during the birth procedure to maximize the quality of the birth tissue procured; and
procuring birth tissue at the birth procedure.

16. The method of procuring birth tissue of claim 15 wherein the trained technician is trained by a third party other than the entity primarily responsible for the health care provided to the birth tissue donor at the birth procedure.

17. The method of procuring birth tissue of claim 16 wherein the trained technician is trained in compliance with applicable tissue collection regulations.

18. The method of procuring birth tissue of claim 17 wherein the trained technician further begins the manufacture of products from the procured birth tissue by engaging in at least one of the following steps:
adding a chemical to the procured birth tissue;
applying a pharmaceutical product to the procured birth tissue;
placing the procured birth tissue in a tissue culturing medium;
marking the procured tissue with a tissue marker; and
dissecting the procured tissues for separate manufacturing and processing steps.

19. The method of procuring birth tissue of claim 17 wherein the trained technician determines the manufacturing path of the procured birth tissue by engaging in at least one of the following steps:
testing the procured birth tissue; and
measuring a physical characteristic of the procured tissue.

20. The method of procuring birth tissue of claim 17 wherein the step of procuring birth tissue at the birth procedure further comprises preserving the procured birth tissue.

21. The method of procuring birth tissue of claim 17 further comprising documenting the procurement of the birth tissue in accordance with applicable regulations.
